(19) United States
(12) Patent Application Publication
(10) Pub. No.: US 2018/0078515 A1
(43) Pub. Date: Mar. 22, 2018

(54) USE OF SURAMIN AND ARGINASE INHIBITORS IN MALIGNANT NEOPLASIA

(71) Applicant: Sammy Oyoo OPIYO, Nairobi (KE)

(72) Inventor: Sammy Oyoo OPIYO, Nairobi (KE)

(21) Appl. No.: 15/558,602

(22) PCT Filed: Apr. 13, 2015
(86) PCT No.: PCT/KE2015/000043

§ 371 Date: Sep. 15, 2017

(30) Foreign Application Priority Data
Mar. 20, 2015 (KE) 2015/002222

(51) Int. Cl. A61K 31/185; A61K 31/17; A61K 45/06

(52) U.S. Cl. CPC A61K 31/185; A61K 45/06; A61K 31/17

(57) ABSTRACT

This is a combination therapy involving use of suramin and its derivatives with Arginase inhibitors to give multifaceted interruption of cancer progression. It also brings immune system restoration and boost to improve disease management. It is applicable also for other conditions like viral and protozoal infections. It entails formulating and administering of this combination in a predetermined dosage and method.

### TECHNICAL FIELD

The present invention relates to management of malignant neoplasia by using a combination therapy comprising an appropriate Suramin salt and an Arginase inhibitor.

### BACKGROUND

Malignant neoplasia is a broad spectrum of diseases involving unregulated cell growth. They are known to spread through the lymphatic system and to succeed, there are six requirements. These are;
1. Sustained proliferative signaling
2. Evasion of growth suppressors
3. Resistance to cell death
4. Enabled replicative immortality
5. Induction of angiogenesis
6. Activated invasion and metastasis

During disease progression, it has been observed there are changes as follows;
1. Suppressed levels of immune cells
2. Decreased ability of T-cells to respond to tumors although the cells remain antigenic.
3. Progressive loss of immuno competence by dendritic cells
4. Other changes generally involving the immune system.

After introduction of Suramin for the treatment of trypanosomiasis, a lot of interest has been developed after laboratory tests showed that it is also effective against various viruses and cancer lines. The challenges faced have been that the demonstrated in vitro activities have not been successfully demonstrated in vivo. These have majorly been due to high dosages used arising from the unfavorably high protein binding nature of the drug.

It has been demonstrated that this protein binding can be reduced and this leads to lowering the dosage requirement for Suramin with improved pharmacokinetics. This approach has also resulted in very negligible toxicity of the drug making it more tolerable to the patients. Apart from disrupting some of the above processes, Suramin has been demonstrated to restore host immune system in various conditions.

Another observation in the cancer disease progression, is the amino acid, arginine metabolism. A further observation is that Arginase inhibitors that modify the Arginine metabolism are able to override the immunosuppressive properties of neonatal cells.

### SUMMARY

It is an object of the present invention, therefore, to formulate a combination therapy comprising an Arginase inhibitor and an appropriate Suramin salt for use in management of malignant neoplasia and other applicable conditions like viral and protozoal infections.

It is yet another object of the present invention to administer the formulation appropriately to achieve the desired pharmacokinetics profile.

Further objects of the invention will become apparent to those skilled in the art from examination of the following detailed description of the invention when taken in conjunction with the appended claims.

### BRIEF DESCRIPTION OF THE INVENTION

In this invention a formulation of low protein binding Suramin salt and an Arginase inhibitor will be administered in very low doses below what is currently in use clinically. The administration will preferably be sublingually but any other route of administration can be deployed. The formulation will preferably be solid or liquid but any other acceptable form will be used if the condition demands.

The sublingual route is preferred due to ease of accessing the lymphatic system. The expected synergism between Suramin and the Arginase inhibitor in terms of immune modulation and anti angiogenesis should be able to retard disease progression especially metastasis through the lymphatic system.

Due to the fact that Suramin has activity against other conditions like viral infections and protozoal infections, it is possible to use this invention in these conditions also.

1. A formulation comprising of Suramin or its derivative and an Arginase inhibitor

2. A combination of claim 1 employing Suramin as a low protein binding salt

3. A combination of claim 2 employing an Arginase inhibitor or a pharmaceutically accepted salt thereof.

4. Use of sufficient amount of a combination of claim 1 for the manufacture of a medicament for the management of neoplasia, viral infections and protozoal infections.
